(19) United States
(12) Patent Application Publication
(10) Pub. No.: US 2023/0176003 A1
(43) Pub. Date: Jun. 8, 2023

(54) ARSENIC DETECTOR AND METHOD OF USE

(71) Applicant: Brewer Science, Inc., Rolla, MO (US)

(72) Inventors: Xi Cao, Tracy, CA (US); Christopher Landorf, Springfield, MO (US); Jonathan J. Fury, Springfield, MO (US); Cody Simmons, Springfield, MO (US); Weston Bowen, Springfield, MO (US)

(21) Appl. No.: 17/856,687

(22) Filed: Jul. 1, 2022

Related U.S. Application Data
(60) Provisional application No. 63/281,783, filed on Nov. 22, 2021.
(60) Provisional application No. 63/217,339, filed on Jul. 1, 2021.

(51) Int. Cl. G01N 27/30; G01N 27/333; G01N 33/18

(52) U.S. Cl. CPC G01N 27/301; G01N 27/333; G01N 33/1813

(57) ABSTRACT

Composites comprising metal-oxide-functionalized carbon nanotubes with metal nanoparticles deposited thereon are provided. These composites can be used as a working electrode in an electrochemical sensor to detect arsenite in aqueous solutions. The composite can electrochemically reduce As³⁺ to As⁰ due to increasing adsorption capability. In one embodiment, Au nanoparticles are deposited on the TiOx/CNT electrode to facilitate the adsorption of As³⁺ on the electrode surface for further electrochemical reduction process. Square wave voltammetry (SWV) is performed to detect the electrochemical reduction of arsenite in water.

### BACKGROUND

### Related Applications

The present application claims the priority benefit of U.S. Provisional Patent Application Ser. No. 63/217,339, filed Jul. 1, 2021, entitled PRINTED ELECTRONIC NANO-CARBON BASED DEVICES AND SYSTEMS TO IMPROVE REAL-TIME SURFACE WATER CONTAMINATION SENSING, and U.S. Provisional Patent Application Ser. No. 63/281,783, filed Nov. 22, 2021, entitled ARSENIC DETECTOR AND METHOD OF USE, each of which is incorporated by reference in their entireties.

### RESEARCH OR DEVELOPMENT

This invention was made with Government support under W912HZ-18-2-0003 Modification P00001 entitled “PRINTED ELECTRONIC NANO CARBON-BASED DEVICES AND SYSTEMS TO IMPROVE REAL-TIME SURFACE WATER CONTAMINATION SENSING,” subaward 18004-001, and under W912HZ-21-2-0019 entitled “QUANTITATIVE WATER SENSING ARRAY FOR RAPID SENSING AND CONTINUOUS MONITORING,” subaward 20206-001, both awarded by the Department of the Army ERDC. The United States Government has certain rights in the invention.

### Field

The present disclosure relates to a sensor and method for detecting arsenic in water.

### Description of Related Art

Arsenic is considered as one of the most toxic elements in the natural environment. The Environmental Protection Agency (EPA) regulates the arsenic standard for drinking water at 10 ppb. In natural water, arsenic generally exists in the form of arsenite (As(III)) and arsenate (As(V)), enhancing its toxicity and mobility. Traditional instrumental analytical methods to identify arsenic in water are complicated and expensive, due to the requirements of high-cost instruments, agent preparation, and well-trained technicians. Therefore, there is a need for high-efficiency, low-cost, and rapid-response sensors for real-time monitoring of the arsenic level in natural water.

### SUMMARY

In one embodiment, the disclosure provides a working electrode comprising carbon nanotubes functionalized with a metal oxide, and metal nanoparticles on the carbon nanotubes, on the metal oxide, or on both of the carbon nanotubes and the metal oxide.

In another embodiment, a sensor is provided, with the sensor comprising a working electrode, where the working electrode comprises carbon nanotubes functionalized with a metal oxide, and metal nanoparticles on the carbon nanotubes, on the metal oxide, or on both of the carbon nanotubes and the metal oxide.

In a further embodiment, the disclosure provides a method of monitoring for the presence of an analyte in water, where the method comprises contacting a device comprising a working electrode with the water to be monitored. The working electrode comprises carbon nanotubes functionalized with a metal oxide, and metal nanoparticles on the carbon nanotubes, on the metal oxide, or on both of the carbon nanotubes and the metal oxide.

### BRIEF DESCRIPTION OF THE DRAWINGS

FIG. 1(A)-(C) is a schematic depiction of one process for making a sensor as described herein;

FIG. 2 is a schematic diagram providing exemplary dimensions of one sensor embodiment described herein;

FIG. 3 is an equivalent circuit diagram for square wave voltammetry;

FIG. 4 is a graph of a square wave voltammetry technique;

FIG. 5 is a view of an example calibration curve;

FIG. 6 is a schematic depiction of one example of a setup for electrochemical techniques such as SWV and chronoamperometry;

FIG. 7(a)-(b) provides SEM images at different magnifications of a working electrode prior to testing as described in Example 4;

FIG. 8(a)-(b) provides SEM images at 25k× (larger image) and 250k× (inset) of electrodeposited Au nanoparticles on a TiOₓ/CNT composite as described in Example 4;

FIG. 9 provides the voltammograms obtained from the calibration dosing test described in Example 6;

FIG. 10 is a graph showing the peak current height (Ip) of each dose plotted vs. the concentration of As in ppb (Example 6);

FIG. 11 is a graph showing the area under the peak current curve (Ap) of each dose plotted vs. the concentration of As in ppb (Example 6);

FIG. 12 provides SWV voltammograms over 20 cycles in 50 ppb arsenite in pH 7, 100 mM phosphate buffer solution, obtained as described in Example 7; and

FIG. 13 is an Anson plot generated as described in Example 8.

### DETAILED DESCRIPTION

The present disclosure is concerned with working electrodes, sensors, and methods of detecting the presence of analytes (e.g., arsenic) in water, preferably in a continuous manner.

### SENSORS

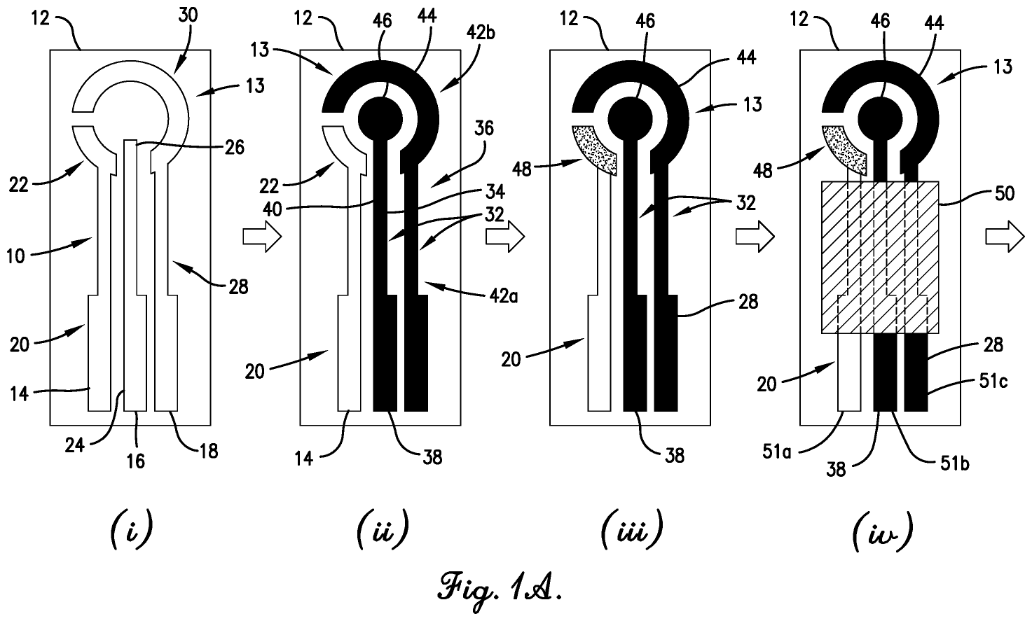

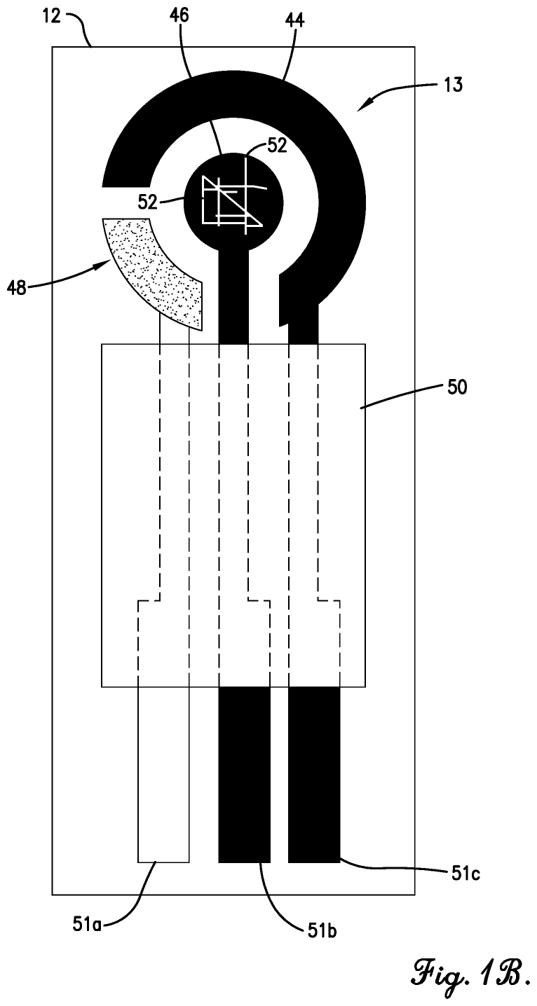

Referring to FIGS. 1(A)-(C), an exemplary sensor formation process is described.

### FIG. 1(A)(i)

Referring to (i) of FIG. 1(A), a current collector layer 10 is deposited on a substrate 12. The substrate 12 may be formed from any number of materials, including those selected from the group consisting of polymers, ceramics, metals, monocrystallines, and combinations thereof. Suitable organic polymers for the substrate 12 include those selected from the group consisting of cyclic olefin polymers (such as those sold as films under the name Zeonor® by Zeon Corporation, with ZEONEX® ZF14-188 being one preferred such film), fluorinated polymers such as polytetrafluoroethylene (“PTFE,” such as those sold as films under the name Teflon® by DuPont), copolymers of tetrafluoroethylene and hexafluoropropylene (“FEP” and “PFA”), polyvinylidene fluoride, polyether ether ketone (“PEEK”), polyetherimide polyphenylene sulfide, polysulfones, polyoxymethylene (“POM”), polyimides, polyamides, polyether sulfones, polyethylene terephthalate (“PET”), polyacrylates, polymethacrylates, polystyrenes, polyesters, polyethylene naphthalate, and combinations of the foregoing.

The substrate 12 preferably has a low water absorbency and low moisture permeability. Preferably, the water absorbency is less than about 3%, more preferably less than about 2%, and even more preferably less than about 1% according to ASTM method D570. It is also preferred that the substrate 12 does not experience hygroscopic expansion or similar deformation, which can generally be determined visually.

The substrate 12 generally has a thickness of about 50 μm to about 5 mm, preferably about 50 μm to about 2.5 mm, more preferably about 75 μm to about 1,000 μm, and even more preferably about 100 μm to about 300 μm. The substrate 12 is preferably planar, or at least presents a planar surface on which current collector 10 is deposited. The substrate may be flexible, but should be rigid enough to enable the appropriate printing and deposition processes. Additionally, substrate 12 is generally rectangular in shape, but could also be configured to be square, circular, etc., as may be desired for the particular application. Substrate 12 is preferably sized and shaped such that the entire current collector 10 can fit on the substrate surface and within the outer perimeter of substrate 12.

The substrate 12 provides a build surface 13 (i.e., a surface on which components of the sensor can be supported during and after sensor construction). The current collector 10 may be deposited on the substrate 12 by any number of conventional techniques, including sputtering, electron beam evaporation, ion-assisted electron beam evaporation, thermal evaporation, inkjet printing, screen printing, gravure printing, or flexography. As illustrated in FIG. 1(A)(i), the deposition method is carried out so as to form an outline of the desired electrode patterns for a reference electrode template 14, a working electrode template 16, and a counter electrode template 18. For example, if the current collector 10 is formed by sputtering, a mask (e.g., a molybdenum mask) having the desired electrode patterns can be used to achieve a current collector 10 with electrode templates 14, 16, and 18 of the desired shape and size.

The size and shape of the electrode templates can vary. In the embodiment illustrated in FIG. 1(A)(i), for example, reference electrode template 14 comprises a generally straight section 20 and a curved end 22. Working electrode template 16 includes a first end 24 and a second end 26, and counter electrode template 18 also includes a generally straight section 28 and a curved end 30. The material from which current collector 10 is formed is chosen so that the current collector 10 exhibits high conductivity. That is, it is preferred that the current collector 10 has a total equivalent series resistance (i.e., as measured by a four point probe or a multimeter) of less than about 5 kΩ, preferably less than about 1000Ω, and more preferably less than about 100Ω. The current collector 10 may be formed of any conductive material that is not oxidized or reduced during device operation, including, but not limited to, gold, silver, platinum, palladium, copper, aluminum, nickel, poly(3,4-ethylenedioxythiophene)-poly(styrene sulfonate), poly(aniline), a carbonaceous material (e.g., carbon, amorphous carbon, carbon nanotubes, graphite, graphene, carbon nanobuds, glassy carbon, carbon nanofibers), and combinations thereof. Oxidation of a material can be tested using Tafel analysis, electrochemical impedance spectroscopy (EIS), or cyclic voltammetry in inert electrolyte solution or other electrochemical methods using inert electrolyte solution, for example. In one especially preferred embodiment, the current collector 10 comprises gold. In another embodiment, the reference electrode template 14, working electrode template 16, and counter electrode template 18 of current collector 10 are made of the same material. In yet another embodiment, the reference electrode template 14 and the counter electrode template 18 are made of the same material, while the working electrode template 16 is formed of a different material. In one embodiment, the current collector 10 comprises a gold layer and is formed by sputtering, thus providing nobility and electrical resistance stability.

Regardless of the material chosen, the average thickness (as measured by an interferometer or stylus profilometer) of the current collector 10 is preferably about 10 nm to about 1,000 nm, more preferably about 50 nm to about 200 nm, and even more preferably about 100 nm.

### FIG. 1(A)(ii)

As shown in (ii) of FIG. 1(A), a protective conductive layer 32 is formed on working electrode template 16 and counter electrode template 18 of current collector 10, thus forming a protected working electrode 34 and a protected counter electrode 36. Protected working electrode 34 has first and second ends 38, 40 while protected counter electrode 36 has first and second portions 42a, 42b. Preferably first portion 42a of protected counter electrode 36 is substantially straight, while second portion 42b is preferably curved, as shown. Collectively, the portion of the current collector 10 forming the counter electrode template 18 at second portion 42b and the portion of the protective conductive layer 32 overlying the counter electrode template at second portion 42b form a counter electrode 44 comprising two layers, with the first counter layer being formed by current collector 10, and the second counter layer being formed from protective conductive layer 32. Protective conductive layer 32 is useful for protecting current collector 10 from detaching from the surface of the substrate 12 when current passes through current collector 10 for an extended time.

As shown in FIG. 1(A)(ii), protective conductive layer 32 is also formed directly on a portion of the substrate 12 (i.e., with no intervening current collector layer 10 between it and substrate 12) at the second end 40 of protected working electrode 34 to form a sensing platform 46 extending into a space partially surrounded by but spaced from the curved end 22 of the reference electrode template 14 and counter electrode 44. The distance between the sensing platform 46 and the reference electrode template 14 and the counter electrode 44 should be as small as possible, provided that it does not create electrical connections between any of the sensing platform 46, the reference electrode template 14, and/or the counter electrode 44.

The protective conductive layer 32 should be chemically inert and exhibit low resistance. That is, protective conductive layer 32 preferably has a sheet resistance of about 1Ω/square/mil to about 5 kΩ/square/mil, more preferably about 1Ω/square/mil to about 1,000Ω/square/mil, and even more preferably from about 1Ω/square/mil to about 500Ω/square/mil.

The protective conductive layer 32 may be formed of any conductive material that is not oxidized or reduced during device operation, including, but not limited to, carbonaceous materials (e.g., carbon, amorphous carbon, carbon nanotubes, graphite, graphene, carbon nanobuds, glassy carbon, carbon nanofibers), gold, platinum, silver, and combinations thereof, with conductive carbon being a particularly preferred material for forming protective conductive layer 32. Oxidation of a material can be tested using Tafel analysis, EIS, or cyclic voltammetry in inert electrolyte solution or other electrochemical methods using inert electrolyte solution, for example. The protective conductive layer 32 may be deposited by any appropriate method, including sputtering, electron beam evaporation, ion-assisted electron beam evaporation, thermal evaporation, inkjet printing, screen printing, gravure printing, or flexography.

When one or more metals are used as the material for the protective conductive layer 32, the protective conductive layer preferably has a sheet resistance of about 1Ω/square/mil to about 5 kΩ/square/mil, more preferably about 1Ω/square/mil to about 500Ω/square/mil. When one or more non-metals, including carbon allotropes or carbon-filled polymers, are used as the material for the protective conductive layer 32, the protective conductive layer preferably has a sheet resistance of about 1Ω/square/mil to about 5 kΩ/square/mil, more preferably about 1Ω/square/mil to about 500Ω/square/mil. Regardless of the material utilized, the average thickness (as measured by a stylus profilometer or four-point probe) of the protective conductive layer 32 is preferably about 1 μm to about 100 μm, more preferably about 5 μm to about 25 μm, and even more preferably about 13 μm.

### FIG. 1(A)(iii)

Referring to FIG. 1(A)(iii), a reference electrode material is deposited as a layer on the curved end 22 of reference electrode template 14, thus forming a reference electrode 48. The reference electrode 48 is formed from any material conventionally used as a reference electrode in 3-electrode cells, including Ag, Ag/AgCl, or combinations thereof. Thus, the reference electrode 48 is preferably a two-layer system, with the first reference layer comprising Ag, Ag/AgCl, or combinations thereof, and the second reference layer comprising the underlying portion of the current collector 10 at the curved end 22 of the reference electrode template 14.

The reference electrode material may be deposited by any conventional means, including stencil printing, screen printing, sputtering, electron beam evaporation, ion-assisted electron beam evaporation, thermal evaporation, inkjet printing, screen printing, gravure printing, or flexography. The average thickness of the reference electrode 48 is preferably about 1 μm to about 100 μm, more preferably about 5 μm to about 25 μm, and even more preferably about 13 μm.

In the embodiment illustrated in FIGS. 1(A)-(C), the reference electrode 48 is fixed to the same substrate 12 as the other components. It will be appreciated that in some embodiments, the reference electrode 48 could be an external reference electrode (i.e., not deposited on substrate 12). The reference electrode 48 can be a real electrode or a so-called pseudo reference electrode.

### FIG. 1(A)(iv)

Referring to FIG. 1(A)(iv), an encapsulant layer 50 is then formed over areas of reference electrode template 14, working electrode template 16, and counter electrode template 18 that are between the ends thereof to protect these areas from being exposed to the analyte. In the illustrated embodiment, encapsulant layer 50 is generally rectangular in shape, although that shape can be altered depending on the area to be protected from analyte contact. Additionally, the encapsulant layer 50 is sized and shaped to encapsulate all of the electrode templates 14, 16, and 18 portions that are to be protected from analyte contact and also to typically be in contact with portions of substrate 12 around and between electrode templates 14, 16, and 18. The encapsulant layer 50 defines the working electrode area precisely and improves measurement reproducibility by leaving counter electrode 44, sensing platform 46 (which will eventually become the final working electrode), and reference electrode 48 unencapsulated for further processing steps and eventual contact with the analyte. The encapsulant layer 50 should be sized to leave uncovered terminal portions of straight section 20 of reference electrode template 14, first end 38 of protected working electrode 34, and straight section 28 of protected counter electrode 36, which will become leads 51a, 51b, and 51c, respectively.

The encapsulant layer 50 should be a dielectric material and preferably has an ionic impedance (measured by electrochemical impedance spectroscopy) of at least about 1 MΩ, preferably at least about 5 MΩ, and more preferably at least about 10 MΩ. The encapsulant layer 50 should have a resistance of at least about 1M Ω, preferably at least about 5 MΩ, and more preferably at least about 10 MΩ. The encapsulant layer 50 must exhibit sufficient adhesion to adjacent layers (including substrate 12) to prevent leakage and/or diffusion of the analyte solution around and/or through the encapsulant layer 50.

The encapsulant layer 50 can be formed from a material chosen from one or more of poly(cycloolefins), polyesters, polyimides, silicones, polyacrylates, polysulfones, and combinations thereof. In one embodiment, the encapsulant is DuPont 5018 dielectric material. In another embodiment, the encapsulant is Zeonex® 790R material. The encapsulant layer 50 may be deposited by any appropriate means, including screen printing, inkjet printing, gravure printing, and flexography. An additional UV cure or baking step may be used to cure the encapsulant layer 50. The average thickness of the encapsulant layer 50 is preferably about 1 μm to about 100 μm, more preferably about 5 μm to about 25 μm, and even more preferably about 13 μm.

### FIG. 1(B)

Referring to FIG. 1(B), metal-oxide-functionalized CNTs are deposited on sensing platform 46 to form a metal oxide-functionalized CNT layer 52. The metal-oxide-functionalized CNT layer 52 may be deposited by electrodeposition, physical vapor deposition (PVD), atomic layer deposition (ALD), screen printing, inkjet printing, spray coating, or spin coating. The average thickness of the metal-oxide-functionalized CNT layer is generally about 1 nm to about 1,000 nm, preferably about 10 nm to about 100 nm, and more preferably about 10 nm to about 50 nm.

The metal oxide-functionalized CNTs can be purchased already functionalized with the desired metal oxide. Alternatively, the functionalized CNTs can be fabricated prior to deposition, with one exemplary process being a two-step process that involves first taking the raw CNTs and polymer wrapping in a pyrene dispersant for non-covalent functionalization. This is an enthalpy-driven interaction forming π-π0 stacking between aromatic rings of CNTs, which when compared to entropic reactions that rely solely on surfactants (such as SDBS), have less aggregation and higher stability. The CNTs are reacted and wrapped with pyrene after placing in chlorosulfonic acid. This mixture is then quenched in water and neutralized with ammonium hydroxide. Next, the solution is filtered leaving the final oxidized CNT paste containing approximately 0.15 wt. % CNTs and 99 wt. % solvent. The remaining mass is residual water and functionalized 1-pyrenemethylamine HCl The CNT paste is then diluted with 2-methyl-1,3-propanediol to prevent the CNTs from drying out as allowing the CNT material to fully dry during filtration or storage will result in the inability to redisperse.

The second step in the CNT fabrication process is the metal oxide functionalization. There are several methods for achieving this including hot pressing of composite powder, pressure-less sintering technique, direct in-situ growth, in situ CVD synthesis, high-intensity ultrasonic radiation, assembling pre-synthesized metal oxide nanoparticles as building blocks on CNTs, spontaneous formation of metal oxide nanoparticles on CNTs, thermal decomposition of metal oxides precursor directly onto the surface of carbon nanotube, hydrothermal crystallization, sol-gel followed by spark plasma sintering, surfactant wrapping sol-gel, chemical precipitation, and controlled heteroaggregation.

Regardless of the functionalization process or if the CNTs are purchased already functionalized, suitable CNTs include single-walled, double-walled, and/or multi-walled CNTs (SWCNTs, DWCNTs, and MWCNTs, respectively).

Preferably, the CNTs are pristine, that is, CNTs having little or no sidewall defects, existing functionalization (other than metal oxide functionalization as described herein, in embodiments where already-functionalized CNTs are purchased), or doping. Suitable CNTs have an onset temperature or initial decomposition temperature when measured by thermogravimetric analysis (TGA) or at least about 400° C. Although the number of walls affects the outer diameter of the CNTs, it is generally preferred that the CNTs used as described herein have an outer diameter of about 0.5 nm to about 20 nm, preferably about 0.6 nm to about 10 nm, and more preferably about 0.7 nm to about 5 nm.

Suitable metal oxides for use in metal-oxide-functionalized CNT layer 52 have a high affinity for As(III) and are excellent localizers for concentrating As(III) near the working electrode, thus improving reduction and oxidation during the stripping process, leading to higher current levels flowing from the working electrode to the counter electrode. Examples of suitable metal oxides for use herein include Fe₃O₄, FeO₂, MnO, CoOx, SnO₂, TiOx, IrO₂, RuOx, and mixtures thereof In one embodiment, oxides of titanium (TiO₁₋₂) are used to functionalize CNT layer 52. The amount of metal oxide utilized is adjusted to optimize the sensitivity of the final device. Too much metal will result in aggregation and loss of surface area/electrical signal, and too little metal will result in less adsorption/electrical signal. The ratio of CNTs to metal oxide is preferably from about 1:300 to about 2:1 by weight, more preferably from about 1:10 to about 1:1 by weight, and even more preferably about 1:2 by weight.

In one embodiment, the metal oxide can be provided as metal oxide nanoparticles. In that instance, the average particle size of the metal oxide nanoparticles will be about 10 nm to about 10 μm, more preferably about 10 nm to about 1 μm, even more preferably about 10 nm to 500 nm, as determined by scanning electron microscope

The use of the functionalized CNTs effectively increases the surface area of the working electrode 56. In this case, the electrochemical surface area (ESA) is preferably greater than the geometrical surface area (GSA). The ESA can be determined experimentally by using well-known electrochemical reactions with a known redox mediator electrolyte solution, such as ferrocene methanol. Using the Anson equation,

Q=nFACD^{1/2}π^{-1/2}t^{1/2}

where Q is the charge in coulombs, n is the number of electrons for oxidation or reduction of one molecule of redox mediator, F is Faraday's constant, A is the electrochemical surface area, C is the concentration of the redox mediator, D is the diffusion coefficient of the solution, typically in cm²/s, and t is time in seconds, the electrochemical surface area, A, can be determined.

The ratio of the ESA to GSA is defined as ρ, the roughness factor. Preferably, the roughness factor of the metal oxide-functionalized CNT layer is at least 1, more preferably at least 1.05, and even more preferably at least 1.1.

### FIG. 1(C)

Finally, and referring to FIG. 1(C), metal nanoparticles 54 are deposited on metal oxide-functionalized CNT layer 52 to form working electrode 56. Thus, the working electrode 56 comprises the portion of the protective conductive layer 32 extending from the second end 26 of the working electrode template 16, to form the sensing platform 46, the sensing platform 46, the metal oxide-functionalized CNT layer 52 on the sensing platform 46, and the metal nanoparticles 54. The metal nanoparticles 54 can be deposited by various existing methods, including electrodeposition (electroplating deposition), physical vapor deposition (PVD), atomic layer deposition (ALD), screen printing, inkjet printing, spray coating, or spin coating. Electroplating deposition is particularly preferred as the potential and deposition time can be adjusted to affect the growth/deposition of the metal nanoparticles. For example, a lower E_{applied} leads to slower growth of the metal nanoparticles while a longer time and/or increased metal nanoparticle levels results in metal nanoparticle clusters with larger cluster size. Alternatively, the metal nanoparticles can be added to the metal-oxide-functionalized CNTs via a chemical reaction, such as by reduction of a metal halide.

Regardless of the preparation method, noble metals are preferred as the nanoparticles because of their high conductivities and chemical inertness. Preferred metal nanoparticles include those chosen from Au, Ag, Pd, Pt, Ru, Ir, and combinations thereof, with Au being particularly preferred.

Additionally, the metal nanoparticles 54 utilized preferably have an average particle size of about 10 nm to about 10 μm, more preferably about 10 nm to about 1 μm, even more preferably about 10 nm to 500 nm as determined by scanning electron microscope. In one preferred embodiment, the ESA of the working electrode 56 with metal nanoparticles 54 will be equal to or greater than the ESA of the working electrode prior to the addition of metal nanoparticles.

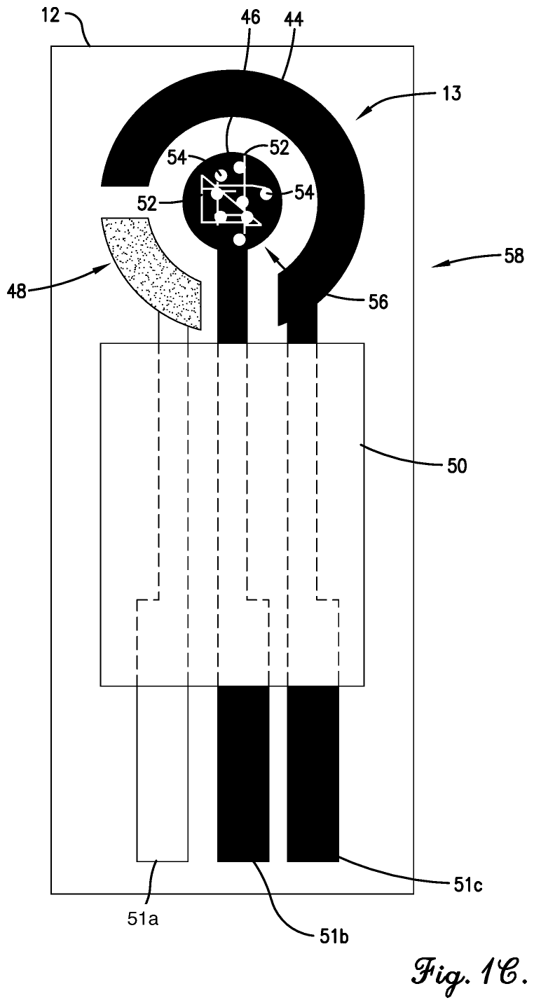

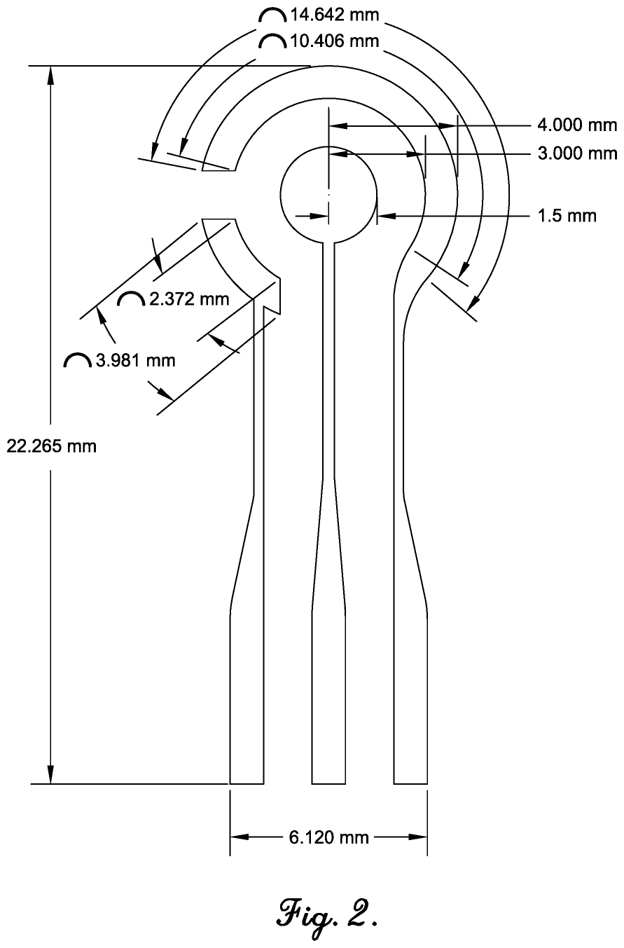

The above process forms the final sensor 58, as shown schematically in FIG. 1(C). FIG. 2 provides exemplary dimensions for one such sensor 58, which is about 22.2 mm×about 6.1 mm. The working electrode 56, counter electrode 44, and reference electrode 48 surface areas are about 7.1 mm², about 67.7 mm², and about 18.4 mm², respectively. However, the sizes of the electrodes can vary.

Importantly, the sensor 58 formed herein is a transducer, which is different from a transistor or electronic switch. It will be appreciated that a transducer, in its simplest definition transforms a signal from one energy form to another energy form, while a transistor in its simplest definition controls the flow of electricity. The latter would include a source (input) and a drain (output), neither of which are present in a transducer.

Advantageously, the sensor 58 can be used for detection of various analytes (e.g., arsenic, and particularly As³⁺). In one embodiment, the sensor 58 can be used as part of the voltammetry system. In another embodiment, the sensor 58 can be used in other devices, and particularly in electrochemical sensor systems. Preferred such systems generally comprise precision microcontroller, a multiplexer array, temperature detector electronics, and a data acquisition system. Additionally, two or more of the sensors 58 can be used in the same system, depending on the user's needs.

In one embodiment, the sensor system comprises a sensing platform for a continuous water resource monitoring by electrochemical detection. Continuous monitoring can be provided for drinking water, fresh water, wastewater, and water produced by reverse osmosis. In one embodiment, the sensor system is placed in a flow path of water to be monitored, so that the water contacts the sensor 58. This device may be used as a standalone sensor in environments where the water parameters (pH temperature, ionic strength) are controlled, or in concert with compensation sensors where water parameters are not controlled. Compensation sensors may include electrical conductivity, temperature, pH, oxidation reduction potential, and/or mass flow. Advantageously, the sensing system is particularly advantageous in low ionic strength environments (<100 mM).

### METHOD OF USE

Voltammetry is one method for quantitative detection of analytes in water. In these systems, the potential is controlled, and current is measured at the working electrode and the counter electrode is the conductor that completes the circuit. The working electrode and counter electrode make up one of the half cells. The other half cell is the reference electrode, which has a constant electrochemical potential, allows no current to flow through it, and is used to measure the working electrode potential. Voltammetry may be implemented in many forms that are well-understood by those having skill in the art, including, but not limited to, linear sweep voltammetry, cyclic voltammetry, and pulse voltammetry techniques (including square wave voltammetry, normal pulse voltammetry, differential pulse voltammetry). The sensor described herein is especially suited for pulse voltammetry, and one especially preferred embodiment is square wave voltammetry.

Square wave voltammetry (SWV) is one of the fastest and most sensitive electrochemical technique for quantitative detection in comparison to the commonly used cyclic voltammetry any other voltametric techniques. Therefore, SWV is used for arsenite detection in water using a three-electrode system and two half-cell reactions. SWV is one of many pulse voltammetry electrochemical techniques. Other pulse voltammetry techniques such as differential pulse, may result in results similar to SWV. In these systems, the potential is controlled, current is measured at the working electrode 56, and the counter electrode 44 is the conductor that completes the circuit. The working electrode 56 and counter electrode 44 make up one of the half cells. The other half cell is the reference electrode 48, which has a constant electrochemical potential, allows no current to flow through it, and is used to measure the working electrode potential.

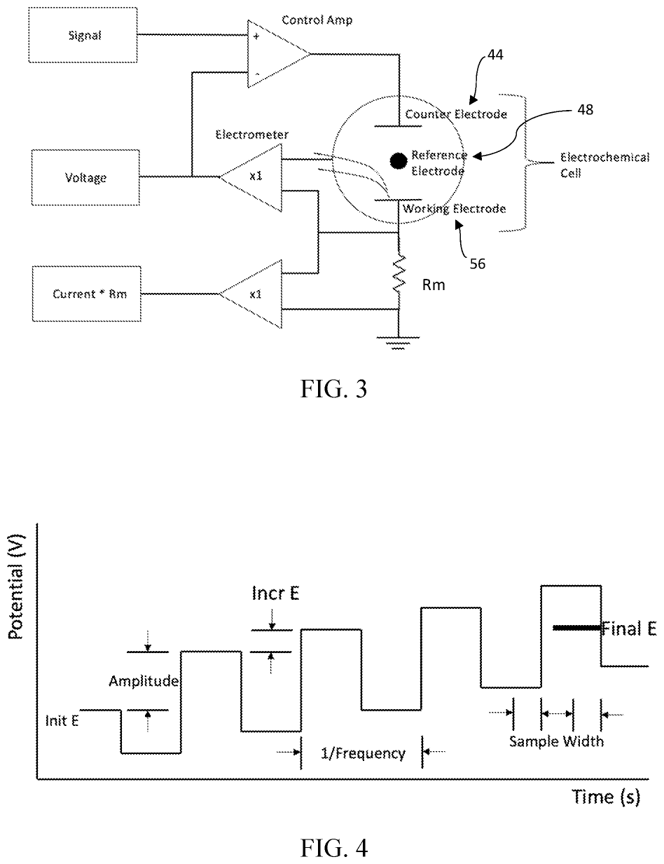

In order to perform SWV on these electrodes, a potentiostat or equivalent circuit shown in FIG. 3 is preferably used. As shown, the “x1” on two of the amplifier indicates that the amplifier is a unity-gain differential amplifier. The output voltage of this circuit is the difference between its two inputs. The blocks labeled “Voltage” and “Current*Rm” are the voltage and current signals that are sent to the system A/D converters (not shown) for digitization. The electrometer circuit measures the voltage difference between the reference and working electrodes (shown as 48 and 56, respectively, in FIG. 1(A)-(C)). The I/E converter measures the cell current and forces the cell current to flow through a current-measurement resistor, Rm. The voltage drop across Rm is a measure of the cell current. The control amplifier compares the measured cell voltage with the desired voltage and drives current into the cell to force the voltages to be the same. The signal circuit is a computer-controlled voltage source that is generally the output of a digital-to-analog (D/A) converter that converts computer generated numbers into voltages.

FIG. 4 illustrates the two steps required for performing SWV using the sensor 58. The first step is the accumulation process. A substantially constant (i.e., +/−0.05 V) voltage (Init E) of −0.5 V vs a Ag/AgCl reference electrode is held on the working electrode 56, causing negative current from the counter electrode 44 to working electrode 56. Here, the arsenite ions are adsorbed and reduced from As³⁺ to As⁰ on the working electrode 56. When plotting current measured versus time, the current should be allowed to stabilize for a certain amount of time. The longer the potential is held, the more As³⁺ that is reduced and the higher the sensitivity of the device. The accumulation process is performed for a time of preferably from about 20 seconds to about 300 seconds, more preferably about 120 seconds, at a voltage of preferably from about −0.8 V to about −0.1 V, more preferably about −0.5 V, particularly in embodiments using titanium-oxide-modified CNTs with gold nanoparticles.

The second step is the stripping process. In this process, the voltage on the working electrode 56 is increased slowly over increments, and preferably maintained substantially constant (i.e., +/−0.05 V) during each increment, to oxidize the As⁰ to As³⁺ off the working electrode 56, allowing ions to flow from the working electrode 56 to counter electrode 44 and generating current through the circuit. The As(III) oxidation peak is generated around 0.2 V. The current generated and measured from this step is the difference between the forward oxidation current and the reverse reduction current. In one cycle of square wave, forward current is measured when going from a negative to positive voltage. The reverse current is measured when going from positive to negative voltage. The difference (forward−reverse) is taken to increase signal and filter out any capacitive current that is generated from a double layer in the sensor 58. This current difference is then plotted on the y-axis against voltage on the x-axis in a line graph to generate a voltammogram with the As(III) oxidation peak around 0.2 V.

A resting step may be performed after each of the first step (the accumulation process) and the second step (the stripping process). This resting step is preferably at least about 1 nanosecond, more preferably about 1 nanosecond to about 1 second, even more preferably about 1 nanosecond to about 100 milliseconds.

There are several parameters in SWV that can be modified to produce better current signal and the most optimal voltammogram. First, the SWV electrochemical voltage window is preferably about −0.5 V to about 0.5 V vs an AgCl reference electrode. The voltage stepping increment is preferably about 0.001 V to about 0.05 V, more preferably about 0.005 V to about 0.025 V, and more preferably about 0.008 V. The frequency of the square wave is preferably about 1 Hz to about 50 Hz, more preferably about 10 Hz to about 40 HS, and more preferably about 25 Hz. Lower frequency allows full transport of the ions and maximum current generation.

Still referring to FIG. 4, the amplitude of the square wave is preferably about 0.001 V to about 0.05 V, more preferably about 0.010 V to about 0.040 V. Lower voltages show the redox currents produced at specific voltages during the voltage incrementing and produce a narrower/sharper current peak when entering the As(III) voltage oxidation range. The sensitivity is preferably about 1×10⁻⁴ A to 1×10⁻⁶ A, and more preferably about 1×10⁻⁵ A. The sensitivity used will determine which resistor in the potentiostat circuit (referred to as Rm above) will be used for measuring current. The current generated from the As(III) oxidation is expected to range from about 0 μA to about 50 μA (particularly in embodiments using titanium-oxide-modified CNTs with gold nanoparticles), depending on the As(III) concentration, so a sensitivity of 1×10⁻⁵ will generally give the most accurate current measurement and show the least amount of noise in the voltammogram. These parameters allow for detection of As(III) at the trace level, with a detection range starting from about 6.2 ppb to about 1,000 ppb, and more preferably starting from about 6.0 ppb, as well as a quantification range starting from about 6.2 ppb to about 1,000 ppb, and more preferably starting from about 6.0 ppb. For example, the detection range in one representative embodiment using titanium-oxide-modified CNTs with gold nanoparticles as the working electrode material is from about 6.2 ppb to about 94 ppb.

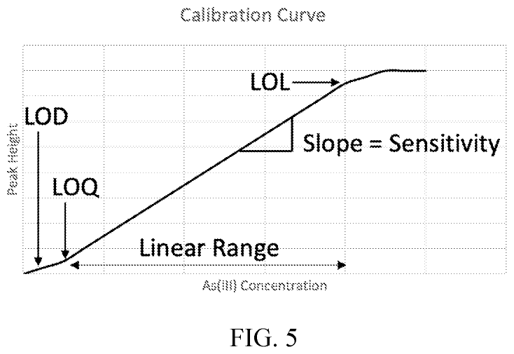

The calibration curve plays an important role in the determination of sensitivity, linear range, and limit of detection (LOD). The calibration test is performed measuring known arsenic concentrations of 0 ppb on the blank cycle up to a predetermined level, such as 100 ppb, after a series of doses. The more doses that are performed, and the smaller the increments, the more data points that will be collected, and the more precise the calibration curve. The voltammograms produced are evaluated for their peak height (Ip) in μA and plotted against As(III) concentration in ppb to generate the calibration curve. An example of a calibration curve is shown in FIG. 5, where the LOD is the lowest quantity of a substance that can be distinguished from the absence of a substance. The limit of quantitation (LOQ) is the lower limit at which two distinct concentrations can be distinguished, the linear range is where the As(III) concentration is proportional to the peak height, the sensitivity is the slope of the linear region, and limit of linearity (LOL) is the upper limit of the linear region where two distinct concentrations can be distinguished.

The linear range can be fit using any suitable technique, such as using a Microsoft Excel software fitting technique to produce the sensitivity or the slope of the line, linear range, and LOD. For example, in one representative embodiment using titanium-oxide-modified CNTs with gold nanoparticles as the working electrode material, the sensitivity was 7e-7 A/ppb, with a linear range from 6.2 ppb to 94 ppb for As(III), and an LOD of 6.2 ppb for As(III).

The calibration curve can then be used to identify the As(III) concentration in an unknown solution. First, the SWV voltammogram is generated using a calibrated sensor 58. Then, the height of the As(III) oxidation peak is measured and compared to the calibration curve. The point along the calibration curve with the same y-value of the peak height observed in the unknown solution is located, and the x-value of that point is the As(III) concentration of the unknown solution.

It will be appreciated that the composite comprising metal-oxide-functionalized CNTs 52 and metal nanoparticles 54 shows excellent catalytic activity for arsenite detection in water with high sensitivity, low limit of detection (LOD), and wide linear range, as described above. This composite can electrochemically reduce As³⁺ to As⁰ due to increased adsorption capability. Taking advantage of the low surface potential and work function, the metal nanoparticles 54 can facilitate the adsorption of As³⁺ on the sensing platform 46 of the working electrode 56 for further electrochemical reduction process.

Additional advantages of the various embodiments will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

### EXAMPLES

The following examples set forth methods in accordance with the disclosure. It is to be understood, however, that these examples are provided by way of illustration, and nothing therein should be taken as a limitation upon the overall scope.

### EXAMPLE 1

### Blank Carbon Device Fabrication

A device was fabricated by using a sputter deposition system (Model SC450, Semicore, CA) to sputter a gold current collector onto a ZEONEX° ZF14-188 substrate (Zeon Europe GmbH, Germany). Deposition was carried out at a rate of 1.0 Å/s for 4 minutes and 20 seconds using a 150 μm molybdenum shadow mask for patterning to achieve a thickness of 100 nm. The patterned substrate was then plasma treated using an AST Products Inc. PS-350 plasma etcher (0.1 Torr chamber pressure, 50 sccm O₂ flowrate, 50 W RF power, and 30 seconds RF time). A conductive carbon material, DuPont BQ242 (DuPont, Circleville, Ohio), was screen printed over the gold current collector using a stainless steel screen (mesh 230 cal with 0.0011″ wire diameter), on an ATMA OE 67 screen printer, fitted with a 70 durometer polyurethane squeegee. The squeegee speed was set to 250 mm/s, and off contact was set to 1.0 mm. Cure was done in a HIX Corporation NP-2410 IR cure oven with a belt speed of 28 inches per minute and temperature of 130° C. The reference electrode material, Sun Chemical-Gwent C2130809D5 (60:40 Ag:AgCl) (Gwent Electric Material Ltd, UK), was screen printed over the gold current collector to form the reference electrode. This screen printing was carried out using a stainless steel screen (mesh 230 cal with 0.0011″ wire diameter), on an ATMA OE 67 screen printer, fitted with a 70 durometer polyurethane squeegee. The squeegee speed was set to 250 mm/s, and off contact was set to 1 mm. Cure was conducted in a HIX Corporation NP-2410 IR cure oven with a belt speed of 28 inches per minute and temperature of 130° C. To define the working electrode area, the material sold under the name DuPont 5018A (DuPont, Circleville, Ohio) was screen printed over the gold current collector using a stainless steel screen (mesh 230 cal with 0.0011″ wire diameter), on an ATMA OE 67 screen printer, fitted with a 70 durometer polyurethane squeegee. The squeegee speed was set to 250 mm/s, and off contact was set to 1.0 mm. Cure was carried in a Heraeus DRS 10/12 UV belt oven with two passes at a belt speed of 4.0 feet per minute.

### EXAMPLE 2

### Preparation of TiOx/CNT Ink

a. Preparation of CNT Dispersion

A 1-liter reactor was loaded with 1.029 grams of Thin-Walled Carbon Nanotubes (Cheaptubes, SKU: 010109) tubes and 1.800 grams of 1-pyrenemethylamine HCl (Sigma Aldrich, St. Louis, Mo.), and the two were mixed thoroughly. A 1-kg bottle of chlorosulfonic acid (Sigma Aldrich, St. Louis, Mo.) was added to the reactor, and the lid was clamped on. The reactor was stoppered and equipped with overhead stirring. The stirring was set at 400 rpm and maintained for 3 days. A 4-liter jacketed quench vessel was equipped with an overhead stirrer and a recirculating chiller set to 5° C., with 1.5 liters of DI water being placed in the reactor. When the DI water reached 5° C., vacuum was used to slowly draw over the chlorosulfonic acid mixture. The temperature was kept between 10° C. and 20° C. by adjusting a release valve on the vacuum line to slow or speed the transfer rate. The temperature was set to 15° C., and a peristaltic pump was used to deliver 1.75 liters of 28-30% ammonium hydroxide (VWR, J. T. Baker®, Batavia, Ill.) at 4.9 mL/min. The resulting mixture was then filtered through an Advantec 3.0 PTFE filter (King Filtration Technologies, St. Louis, Mo.). The vacuum timer was set for 8 hours. The wet black solid was removed from the filter and mixed with a solution of 200 mL of 28-30% ammonium hydroxide in 1,800 mL of DI water. The filtration and addition of ammonium hydroxide were then repeated. The resulting solid was again filtered through an Advantec 3.0 μM, PTFE filter, and the black solid was then dispersed in 600 grams of 2-methyl-1,3-propanediol (Sigma Aldrich, St. Louis, Mo.). The final oxidized CNT paste contained 0.167 wt. % CNTs and 99% wt. % solvent, with the remainder being residual water and functionalized 1-pyrenemethylamine HCl. Next, 2.645 grams of this material were dispersed into 250 mL of DI water by sonication. The absorbance at 550 nm was measured. Multiplying the resulting number by the dilution factor gave an OD of 67.06.

b. Functionalizing CNTs with TiOx and Forming Ink

The dispersion in 2-methyl-1,3-propanediol was then diluted to an OD of 30 with 2-methyl 1,3-propanediol using a planetary mixer set at 1,350 rpm revolutions and 1,350 rpm rotation. Next, 200 grams of the OD=30 material and 50 grams of 2-methyl-1,3-propanediol were added to a 1-liter reactor equipped with overhead stirring and flushed with nitrogen. The stir rate was set to 450 rpm, and 100 grams of 2-methyl-1,3-propanediol and 3.62 mL of a 50% solution of titanium(IV) bis(ammonium lacto)dihydroxide in water (Sigma Aldrich, PN: 388165) were added to a 250-mL Schlenk flask connected to nitrogen. The contents of the flask were stirred with a magnetic stir bar and flushed for 5 minutes. Using an air-tight syringe, 50 mL of the solution were withdrawn from the flask, placed in a syringe pump, and added to the stirred reaction (in the 1-L reactor containing the OD=30 material) at 120 mL/hr. When solution addition was finished, the 1-L reactor was fitted with a reflux condenser and heated to 115° C. for 180 minutes. The reactor contents were then collected in a 300-gram planetary mixer jar.

### EXAMPLE 3

### Spray Coating TiOx/CNT Ink onto Carbon Devices

An automated, programmable coating system (sold under the name ExactaCoat, Sono-Tek Corporation, Milton, N.Y.), was used to spray coat the TiOx/CNT ink from Example 2b onto the device from Example 1. While preheating the coating system tray to 125° C., the TiOx/CNT (further diluted to 0D=2 with 2-methyl 1,3-propanediol) dispersion was probe sonicated for 30 minutes after which the CNT dispersion was loaded into a 50-mL syringe and secured to the coating system's syringe pump, whose rate was set to 500 μL/min. The preheated tray was taken out, and a wafer was placed in the middle of the tray. The wafer was covered with a metal stencil cutout, so that only the working electrodes were visible, with magnets used to hold the stencil in place. The tray was placed back into the coating system and allowed to heat for 5 minutes. The spray coating parameters were set up by opening the PathMaster software and setting the syringe pump rate to 500 μL/min and the shaping air pressure to 0.6 kPa. The arm position/speed parameters were programmed, using the area command with 3 different sets of X, Y, and Z coordinates. These coordinates were set manually by moving the arm and teaching the coordinates for the bottom, top, and right side of the wafer. Z was constant at 57.0150 mm. The resulting XYZ coordinates were 48.5550, 38.600, 57.0150 for the bottom left corner of the wafer, 234.1150, 38.3600, 57.0150 for the top left corner, and 234.1150, 202.9100, 57.0150 for the top right corner. The Path Speed was set to 90 mm/s, and the Area Spacing set to 2. Once these parameters were set up, spray coating was initialized, and the working electrodes were coated with the ink, forming a TiOx/CNT composite layer on the working electrodes

### EXAMPLE 4

### Electrodeposition of Au Nanoparticles on TiOx/CNT Composite Layer

A stock solution of 0.1M HAuCl₄ and 0.1M K₂SO₄ was prepared by adding 33.979 grams of HAuCl₄, 17.43 grams of K₂SO₄, and 200 mL of DI water to a 1-L volumetric flask. After the salts were totally dissolved, the flask was filled to the graduation marking of the volumetric flask with the DI water. After thoroughly stirring, the stock solution was ready to use.

For electrodeposition of Au nanoparticles, about 18 mL of the stock solution was transferred into a 20-mL PTFE vial, and the TiOx/CNT-coated device was properly connected with working electrode and counter electrode and placed into the solution. A homemade Ag/AgCl reference electrode made from a glass pipette, silver wire, and supersaturated KCl solution in agarose gel served as the reference electrode. The chronoamperometry was performed by a CHI660E potentiostat along with a CHI684 multiplexer (both from CH Instructions, Inc.).

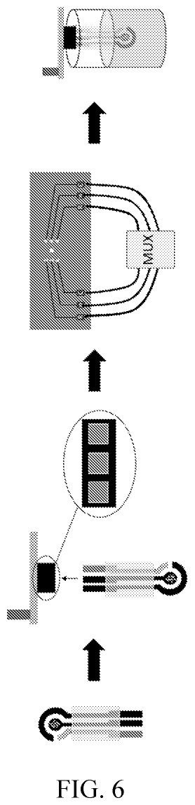

A schematic illustration of this setup is shown in FIG. 6. The sensor or sensors (up to 2) were attached to a custom printed circuit board (“PCB”) obtained from Osh Park (Portland, Oreg.). The potentiostat MUX wires were attached to metal pin connectors corresponding to the correct electrode. The electrodes were then placed in solution at a depth where just the portions of the three electrodes were exposed. The parameters were initial voltage: −1.0 V, high voltage: −1.0 V, low voltage: −1.02 V, and pulse width: 100 seconds.

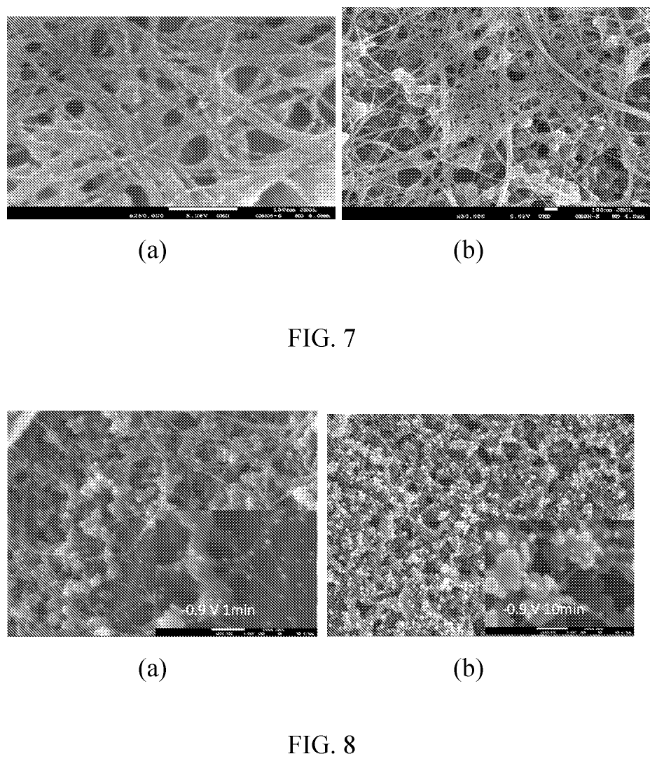

Scanning electron microscope images were taken of the working electrode before voltage application commenced for comparison purposes. FIG. 7(a) shows an image at 250 k× magnification while FIG. 7(b) shows one at 50 k× magnification, with the 100-nm scale being shown at the bottom of each image as a white horizontal line. FIGS. 8(a) and (b) show images at 25 k× magnification, with each including an inserted image at 250 k× magnification. FIG. 8(a) was taken at 1 minute after voltage application was commenced, while FIG. 8(b) was taken at 10 minutes after voltage commencement. In these figures, the CNTs are observed as long strands with clumps of TiO₂, while the Au nanoparticles are small dots covering the surfaces of the CNTs. Thus, examining and comparing FIGS. 7 and 8 confirms the presence of Au nanoparticles on the TiOx/CNT-coating.

### EXAMPLE 5

### Preparation of Gold-functionalized TiOx/CNT Composite

A dispersion prepared as described in Example 2a was diluted to an OD of 30 with 2-methyl 1,3-propanediol using a planetary mixer set at revolution: 1,000 rpm, rotation: 300 rpm, time: 60 seconds, wave: on, vacuum: on. Next, 200 grams of the OD=30 material and 25 grams of 2-methyl-1,3-propanediol were added to a 1-liter reactor with an overhead mixer and reactor lid secured on the reactor. The reactor was lowered into an oil bath and stirred at 450 rpm. A ¼″ PFA tubing was attached to nitrogen through a 24/40 adapter, and the reaction mixture was flushed with nitrogen.

Next, 100 grams of 2-methyl-1,3-propanediol were added to a 250-mL Schlenk flask. A stir bar was added to this flask, and it was connected to the nitrogen line, with the nitrogen flow rate being set to 1 CFM. The nitrogen valve to the reactor was then closed and stoppered. The stir rate was set to 450 rpm, and Schlenk flask contents were stirred for 5 minutes to allow the flask to clear out humid air. After 5 minutes of stirring, 3.616 mL of a 50% solution of titanium(IV) bis(ammonium lactate)dihydroxide in water (Sigma Aldrich, PN: 388165) were added to the Schlenk flask. A piece of ¾″ PFA tubing was then attached to a 50-mL airtight syringe, and that syringe was used to remove 50 mL of the titanium mixture from the Schlenk flask. A Universal 24/40 to hose inlet with locking ring (Chemglass, CG-1047-05) was placed on the reactor. The syringe was placed in a syringe pump having a tube through the adapter, and the mixture was added to the 1-L reactor containing the OD=30 material at a pump rate of 120 mL/hr. When the addition was complete, the adapter was replaced with a condenser open to air. The temperature of the oil bath was set to 115° C., and the reaction was allowed to stir at 180 rpm for 180 minutes. After the reactor cooled back to room temperature, the condenser was removed, and 0.35 grams of gold(III) chloride hydrate (Sigma Aldrich, St. Louis, Mo.) were added to the reactor while it was stirring. Next, a solution was prepared by mixing 0.25 gram of tetrabutylammonium cyanoborohydride (Sigma Aldrich, St. Louis, Mo.) with 25 mL of 2-methyl-1,3-propanediol with a magnetic stirrer until the tetrabutylammonium cyanoborohydride was dissolved. The resulting solution was placed in a 25-mL syringe equipped with ¼″ PFA tubing and a Universal 24/40 to hose inlet with locking ring (Chemglass, CG-1047-05) and added to the reaction mixture in the 1-L reactor at a rate of 50 mL/hr through the 1/4″ tubing while stirring at 380 rpm. Stirring was continued overnight at 180 rpm and under nitrogen, after which the material was collected, and a UV-Vis spectrum was taken, which showed a titanium oxide peak. The reaction mixture was placed in a glass 1-liter wide-mouth bottle, which was filled with acetone (Sigma-Aldrich Inc., St. Louis, Mo.). This was processed using a high sheer mixer for 1 minute at 10,000 rpm and filtered using a 3.0-μm Advantec PTFE filter, not allowing the vessel to go completely dry. This resulting solid was again washed with 1 liter acetone, followed by 1 liter of 2-propanol, followed by 1 liter of water.

### EXAMPLE 6

### Evaluation of Calibration Curve of Arsenic Sensor

To evaluate the calibration curve of the TiOx/CNTs with Au-electrodeposited nanoparticles, a base solution of 100 mM phosphate buffer with pH 7 was prepared. This was done by adding 15.487 grams of K₂HPO₄·7H₂O (Sigma-Aldrich Inc., St. Louis, Mo.) and 5.827 grams of K₂HPO₄·H₂O (Sigma-Aldrich Inc., St. Louis, Mo.) into 800 mL of DI water. The solution was adjusted to pH 7 through titration using a 0.1M NaOH solution (Sigma-Aldrich Inc., St. Louis, Mo.) and then topped off to 1,000 mL with DI water. A stock solution of 0.02 mM NaAsO₂ was prepared by first adding 2.5 mL of 0.05M NaAsO₂ (Sigma-Aldrich, St. Louis, Mo.) solution using a Thermo Scientific Finnpipette into a 1.0-L volumetric flask and filled with phosphate buffer solution. This 0.02 mM NaAsO₂ and 0.1M phosphate buffer pH 7 solution was used for dosing in the calibration test.

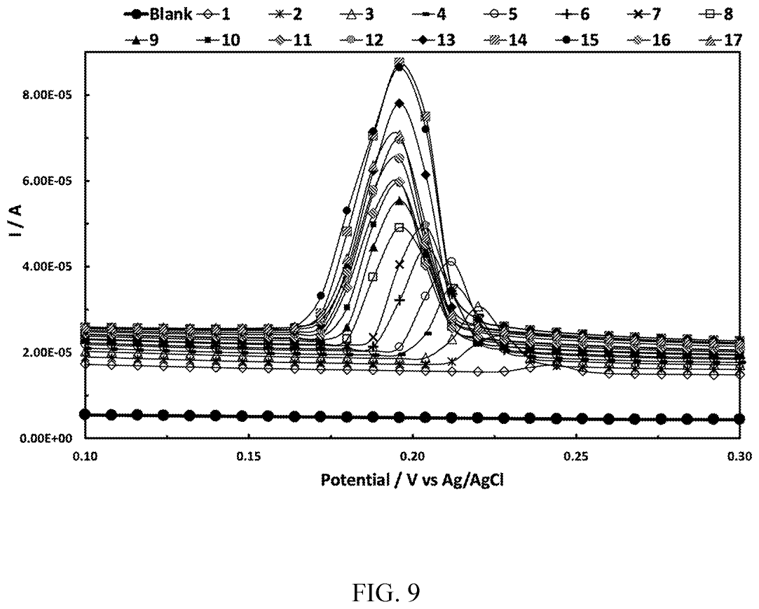

The calibration test was performed by dosing a pH 7, 100 mM phosphate buffer solution with small increments of 0.02mM NaAsO₂ and 0.1M phosphate buffer solution via a pipette to give arsenic concentrations of 0 ppb on the blank cycle and up to 94 ppb after 16 doses. Five minutes were allowed between doses to stir and allow the solution in the vial to homogenize. The SWV was performed using a CHI660E potentiostat and parameters used were initial voltage: −0.5 V vs. 3M Ag/AgCl, final voltage: 0.5 V vs. 3M Ag/AgCl, step increment: 0.008 V, amplitude: 0.025 V, accumulation time: 300 s, frequency: 25 Hz, and sensitivity for current measurement: 1e-5. The voltammograms of every cycle were plotted together and are shown in FIG. 9. As can be seen, the As⁰ oxidation peak started at 0.244 V and drifted to 0.196 V after 16 doses. This is confirmed to be the As⁰ oxidation peak as a blank cycle without arsenite didn't show any peaks.

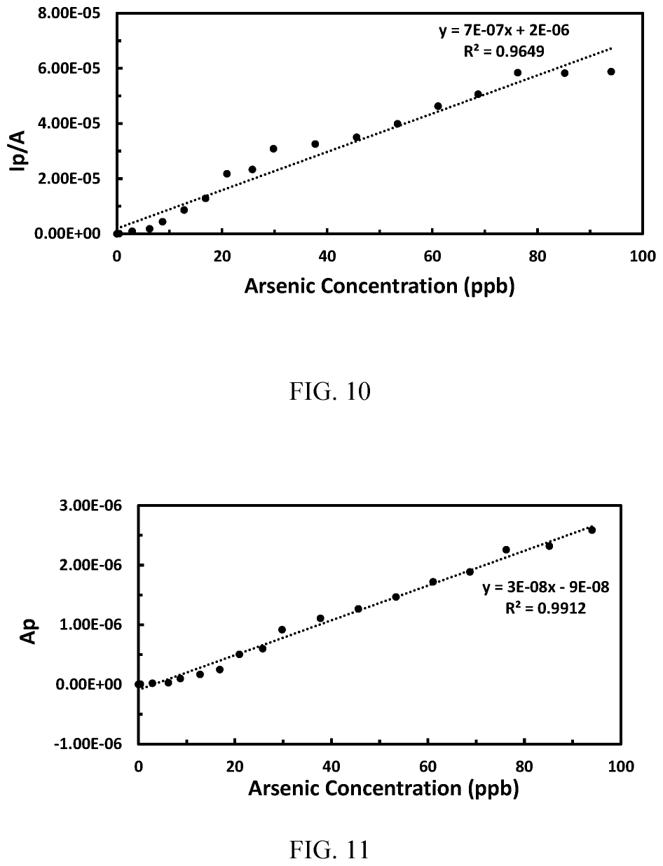

The calibration curves were obtained by plotting the arsenite oxidation peak height (Ip) or peak area (Ap) respectively, as shown in FIGS. 10 and 11. A linear trend was observed from 0 to 94 ppb for the Ap vs. As concentration graph with a slope of 3e-8 A.U./ppb, which is considered as the sensitivity. A similar trend was noticed with Ip vs. As concentration as well with a slope (sensitivity) of 7e-7 A/ppb. The limit of detection (LOD) was defined after the second dose at a concentration of 6.2 ppb arsenite in water. The linear range was from 6.2 to 94 ppb arsenite for the Au-functionalized TiOx/CNT composite.

### EXAMPLE 7

### Evaluation of Long-Term Stability of Arsenic Sensor

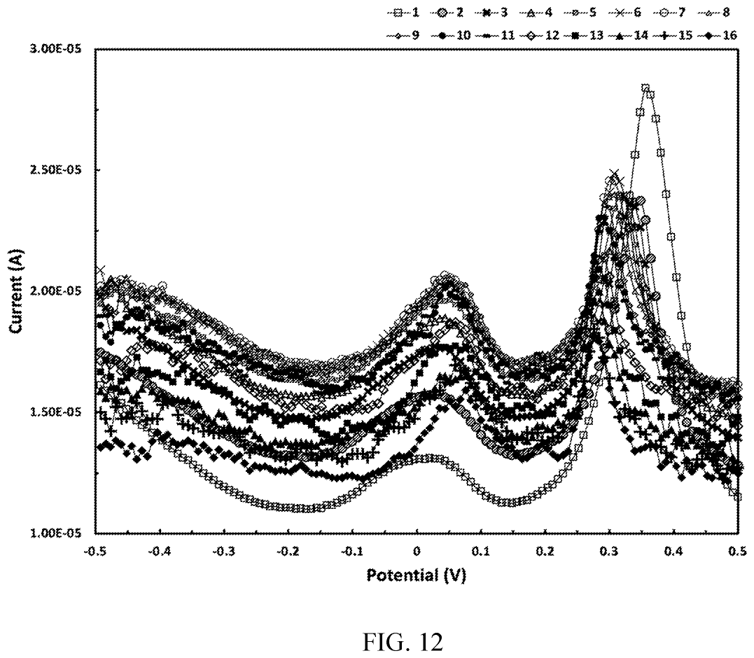

To evaluate the long-term stability of the Au-functionalized TiOx/CNT composite towards arsenite detection in water, square wave voltammetry was performed using a sensor having Au-functionalized TiOx/CNTs on the working electrode in 100 mM phosphate buffer pH 7 with 50 ppb arsenite for 16 cycles with one SWV measured every 1.5 hours. The solution was stirred with a stir bar (10 mm length, 3 mm diameter) at 450 rpm. The SWV parameters were held constant as follows: initial voltage: −0.5 V vs. Ag/AgCl, final voltage: 0.5 V vs. Ag/AgCl, step increment: 0.008 V, amplitude: 0.025 V, accumulation time: 300 seconds, frequency: 25 Hz, and sensitivity for current measurement: 1e-5. All 20 SWVs were plotted together as shown in FIG. 12. Ideally, the peak current height should be the same for all 20 cycles indicating the arsenic sensors are stable for 20 cycles. FIG. 12 indicates that the Au-functionalized TiOx/CNT composite did show excellent stability over 20 cycles.

### EXAMPLE 8

### Determination of Electrochemical Surface Area

In this Example, 1 mM ferrocene methanol in 100 mM KCl was used as the solution. Chronocoulometry was used with 0 mV and 500 mV vs. homemade Ag/AgCl (Sat. KCl) reference electrodes using 2-s pulse widths. The diffusion coefficient of the redox mediator solution was calculated using cyclic voltammetry and the Randles-Ševčík equation.

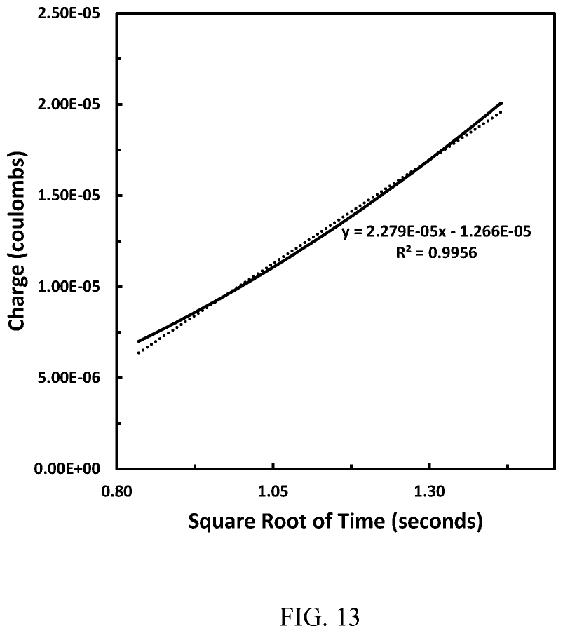

An example of an experimental Anson plot is shown in FIG. 13. The slope of this plot was used to calculate the ESA of a bare CNT electrode. The diffusion coefficient of 1 mM ferrocene methanol in 100 mM KCl was approximately 6.43909×10-6 cm²/sec. The GSA of the CNT film was approximately 7.75 mm² and the ESA calculated from this experiment using the Anson equation was 8.24 mm². This means that this CNT electrode had a roughness factor of about ρ=1.06. Typically, a larger roughness factor would be expected but since such a small amount of CNTs was spray coated onto the substrate, there may have been nonconductive sections of the CNT film, which would decrease the ESA and, therefore, the calculated roughness factor.

We claim:

1. A working electrode comprising:
carbon nanotubes functionalized with a metal oxide; and
metal nanoparticles on said carbon nanotubes, on said metal oxide, or on both of said carbon nanotubes and said metal oxide.

2. The working electrode of claim 1, wherein said metal oxide is chosen from TiOx, Fe₃O₄, FeO₂, MnO, CoOx, SnO₂, IrO₂, RuOx, and mixtures thereof.

3. The working electrode of claim 1, wherein said metal nanoparticles are chosen from Au, Ag, Pd, Pt, Ru, Ir, and mixtures thereof.

4. The working electrode of claim 1, wherein said metal oxide comprises TiOx.

5. The working electrode of claim 1, wherein said metal nanoparticles comprise Au.

6. The working electrode of claim 1, further comprising a substrate presenting a build surface, said working electrode being supported on said build surface.

7. The working electrode of claim 6, wherein said substrate is formed from a material comprising one or more of polymers, ceramics, metals, or monocrystallines, wherein said polymer is chosen from cyclic olefin polymers, fluorinated polymers, tetrafluoroethylene and hexafluoropropylene copolymers, polyvinylidene fluoride, polyether ether ketone, polyetherimide polyphenylene sulfide, polysulfones, polyoxymethylene, polyimides, polyamides, polyether sulfones, polyethylene terephthalate, polyacrylates, polymethacrylates, polystyrenes, polyesters, polyethylene naphthalate, or mixtures thereof.

8. The working electrode of claim 6, further comprising a current collector layer on said build surface.

9. The working electrode of claim 8, wherein said current collector layer comprises gold, silver, platinum, palladium, copper, aluminum, nickel, poly(3,4-ethylenedioxythiophene)-poly(styrene sulfonate), poly(aniline), a carbonaceous material, or mixtures thereof.

10. The working electrode of claim 8, wherein said current collector layer presents an upper surface remote from said build surface, and said working electrode further comprises a protective conductive layer on said upper surface, said protective conductive layer presenting a first surface remote from said upper surface, and said carbon nanotubes being on said first surface.

11. The working electrode of claim 10, wherein said protective conductive layer comprises a carbonaceous material, gold, platinum, silver, or mixtures thereof.

12. The working electrode of claim 10, further comprising an encapsulant layer over a portion of said first surface.

13. The working electrode of claim 12, wherein said encapsulant layer comprises poly(cycloolefins), polyesters, polyimides, silicones, polyacrylates, polysulfones, or mixtures thereof.

14. A sensor comprising a working electrode according to claim 1.

15. The sensor of claim 14, further comprising a counter electrode and a reference electrode.

16. A sensor comprising the working electrode according to claim 6, further comprising a counter electrode and a reference electrode, wherein said counter electrode is on said build surface of said substrate.

17. The sensor of claim 16, wherein said reference electrode is on said build surface of said substrate.

18. The sensor of claim 15, wherein said counter electrode comprises:
a first counter layer comprising gold, silver, platinum, palladium, copper, aluminum, nickel, poly(3,4-ethylenedioxythiophene)-poly(styrene sulfonate), poly(aniline), a carbonaceous material, or mixtures thereof; and
a second counter layer adjacent said first counter layer, said second counter layer comprising a carbonaceous material, gold, platinum, silver, or mixtures thereof.

19. The sensor of claim 15, wherein said reference electrode comprises silver, silver chloride, or mixtures thereof.

20. The sensor of claim 19, wherein said silver, silver chloride, or mixture thereof is part of a first reference layer, and said reference electrode further comprises a second reference layer adjacent said first reference layer and comprising gold, silver, platinum, palladium, copper, aluminum, nickel, poly(3,4-ethylenedioxythiophene)-poly(styrene sulfonate), poly(aniline), a carbonaceous material, or mixtures thereof.

21. A method of monitoring for the presence of an analyte in water, wherein said method comprises contacting a device comprising the working electrode of claim 1 with the water to be monitored.

22. The method of claim 21, wherein said analyte is arsenic ions.

23. The method of claim 21, wherein said device is capable of detecting arsenite present in water at levels of about 6 ppb.

24. The method of claim 21, wherein said contacting comprises positioning said device within a flow path of the water to be monitored.

25. The method of claim 21, wherein the device further comprises a reference electrode comprising Ag/AgCl, further comprising applying a substantially constant initial voltage of about −0.8 V to about −0.1 V vs. the reference electrode to said working electrode for about 20 seconds to about 300 seconds during said contacting, said applying causing As³⁺ to be reduced to As⁰.

26. The method of claim 25, further comprising removing said initial voltage to provide a resting period of about 100 milliseconds to about 1 second.

27. The method of claim 25, further comprising applying a second voltage to said working electrode to cause at least some As⁰ to oxidize to As³⁺, said second voltage being about 0.001 V to about 0.05 V vs. Ag/AgCl greater than said initial voltage.

28. The method of claim 27, further comprising removing said second voltage to provide a resting period of about 1 nanosecond to about 100 milliseconds.

29. The method of claim 27, further comprising applying incrementally increasing voltages to said working electrode each followed by a resting period until said voltage is at least about 0.5 V vs. Ag/AgCl.

30. The method of claim 29, wherein the difference in oxidation and reduction currents is observed during said applying and resting so as to identify an oxidation peak, further comprising comparing that peak to a calibration curve to determine the As³⁺ concentration in said water.
